(19) United States
(12) Patent Application Publication
(10) Pub. No.: US 2015/0148688 A1
(43) Pub. Date: May 28, 2015

(54) NEURAL PROBE ARRAY HAVING WAVEGUIDE MEMBER WITH IMPROVED WAVEGUIDE CHARACTERISTICS AND MANUFACTURING METHOD THEREOF

(71) Applicant: KOREA INSTITUTE SCIENCE AND TECHNOLOGY, Seoul (KR)

(72) Inventors: Il-Joo CHO, Seoul (KR); Eui Sung YOON, Seoul (KR); Hyunjoo Jenny LEE, Seoul (KR); Yoo-jin SON, Seoul (KR)

(21) Appl. No.: 14/312,799

(22) Filed: Jun. 24, 2014

(30) Foreign Application Priority Data
Nov. 25, 2013 (KR) 10-2013-0144043

(51) Int. Cl. A61B 5/00; A61B 1/07

(52) U.S. Cl. CPC A61B 5/0084; A61B 1/07; A61B 5/4064
USPC 600/478; 29/428

(57) ABSTRACT

A neural probe array includes a probe body that is implanted into a subject, a fixture body to support a rear end of the probe body, a cladding extending in a lengthwise direction of the probe body in an upper part of the probe body, and an optical waveguide member installed on the cladding along the cladding, and the cladding is embedded in a recessed cavity formed in the upper part of the probe body. A method for manufacturing the neural probe array includes forming the cavity in an upper part of a first substrate, embedding the cladding in the cavity, forming the optical waveguide member on the cladding along the cladding, and forming the probe body by cutting the first substrate off.

### CROSS-REFERENCE TO RELATED APPLICATION

This application claims priority to Korean Patent Application No. 10-2013-0144043, filed on Nov. 25, 2013, and all the benefits accruing therefrom under 35 U.S.C. §119, the contents of which in its entirety are herein incorporated by reference.

### BACKGROUND

1. Field

The present disclosure relates to a neural probe array and a manufacturing method thereof, and more particularly, to a neural probe array having an optical waveguide member with significantly improved light guiding efficiency that applies an optical stimulus to neurons using the optical waveguide member and receives a response thereto, and a manufacturing method thereof.

[Description about National Research and Development Support]

This study was supported by the Original Technology R&D Project for Brain Science of Ministry of Education and Science Technology, Republic of Korea (Project No. 1345191100) under the superintendence of National Research Foundation of Korea.

2. Description of the Related Art

Recently, studies are actively being conducted to investigate how nerves work by stimulating neurons in a subject and sensing and analyzing a signal in response thereto.

To stimulate neurons in a subject directly and receive information in response thereto, an implantable neural probe is being used. Also, to detect as much information as possible in response to brain neuron stimulation, an ultra-micro neural probe with an integrated electrode array has been developed.

Among conventional neural probes, some apply an electrical stimulus to neurons using an integrated electrode in a probe body. However, there are drawbacks in that neurons may be damaged when an electrical stimulus is applied to the neurons, and local stimulation to a desired part is impossible because constituent substances of neurons are electrically conductive.

Thus, recently, a method that applies an optical stimulus to neurons using light and receives a signal in response thereto is being introduced.

According to an example of a related art, an optical stimulation probe designed to apply an optical stimulus has an optical fiber attached to a silicon probe body directly and is implanted into a subject. In this case, there are problems with imprecise control of a stimulation site and an increase in probe size.

In relation to this, a neural probe array with an optical waveguide member for light transmission has been proposed.

A neural probe array with an optical waveguide member achieves probe body minimization by attaching an optical fiber having a relatively large diameter, rather than to a probe body, to a fixture body used to fix the probe body.

Specifically, an optical waveguide member is attached to a probe body to receive light from an optical fiber and transmit it to neurons. A cladding is attached between the optical waveguide member and the probe body to allow for total internal reflection of light having passed through the optical waveguide member.

The fact that as a cladding becomes thicker, total internal reflection of an optical waveguide member increases and a loss of light reduces is known.

However, according to the related art, because the cladding is patterned on the probe body at a predetermined thickness and the optical waveguide member is patterned on the cladding, the probe body increases in overall thickness.

On the other hand, if the cladding reduces in thickness to reduce the overall thickness of the probe body, the loss of light greatly increases.

### SUMMARY

The present disclosure is designed to solve the above problem of the related art, and therefore, the present disclosure is directed to providing a neural probe array that may greatly increase light transmission efficiency through an optical waveguide member while reducing an overall thickness of a probe body, and a manufacturing method.

To achieve the object, according to one aspect, there is provided a neural probe array including a probe body that is implanted into a subject, a fixture body to support a rear end of the probe body, a cladding extending in a lengthwise direction of the probe body in an upper part of the probe body, and an optical waveguide member installed on the cladding along the cladding, wherein the cladding is embedded in a recessed cavity formed in the upper part of the probe body.

According to an embodiment, the neural probe array may further include an optical fiber fixed in the fixture body to transmit light to the optical waveguide member, wherein the cavity is formed to extend to a front end of the optical fiber across an upper part of the fixture body so that the cladding and the optical waveguide member extend to the front end of the optical fiber, and a rear end of the optical waveguide member is aligned with the front end of the optical fiber to allow light transmission with the optical fiber.

Also, a plurality of probe bodies may be formed in one fixture body, and the optical waveguide member may extend as one strand from the front end of the optical fiber and branch into a plurality of strands which may extend to each of the plurality of probe bodies.

Also, the cladding may be completely embedded in the cavity without protruding beyond a top of the probe body.

In this instance, the cladding may be formed with a same height as the cavity.

According to another aspect, there is provided a method for manufacturing the neural probe array including forming the cavity in an upper part of a first substrate, embedding the cladding in the cavity, forming the optical waveguide member on the cladding along the cladding, and forming the probe body by cutting the first substrate off.

According to an embodiment, the embedding of the cladding in the cavity may include forming a second substrate having a lower softening point than the first substrate, bonding the second substrate onto the first substrate, applying a higher temperature than the softening point of the second substrate to the first substrate and the second substrate to fill the cavity with the melted second substrate, and removing the other portion than a portion of the second substrate filled in the cavity.

In this instance, the bonding of the second substrate onto the first substrate may be performed in a vacuum state so that the cavity is sealed in a vacuum state by the second substrate, and the filling of the cavity with the second substrate may be performed in a non-vacuum state, and the second substrate may be sucked into the cavity by a pressure difference between an inside and an outside of the cavity.

According to an embodiment, the probe body and the fixture body may be integrally formed by cutting the first substrate off, the method for manufacturing the neural probe array may further include forming, in the fixture body, a groove in which the optical fiber for transmitting light to the optical waveguide member is seated, and attaching the optical fiber to the groove, the cavity may be formed to extend to a front end of the optical fiber across the upper part of the fixture body so that the cladding and the optical waveguide member extend to the front end of the optical fiber, and a rear end of the optical waveguide member may be aligned with the front end of the optical fiber to allow light transmission with the optical fiber.

### BRIEF DESCRIPTION OF THE DRAWINGS

FIG. 1 is a perspective view illustrating a neural probe array according to an exemplary embodiment.

FIG. 2 is a diagram illustrating a process of manufacturing a neural probe array according to an exemplary embodiment.

FIG. 3 is a cross-sectional view illustrating a stack structure of a neural probe array according to an exemplary embodiment.

FIG. 4 is a graph illustrating light transmission efficiency of a neural probe array according to an exemplary embodiment.

### DETAILED DESCRIPTION

Hereinafter, preferred embodiments are described with reference to the accompanying drawings. While the present disclosure is described with reference to exemplary embodiments shown in the drawings, it is intended that the embodiments are merely described as a mode for carrying out this disclosure and the scope of the present disclosure and its essential elements and functions are not limited by such embodiments.

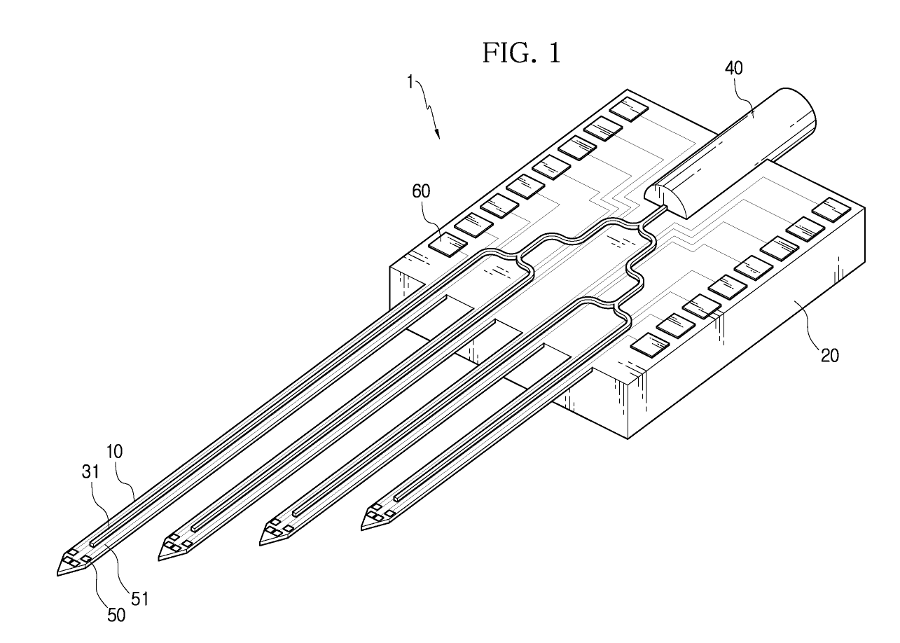

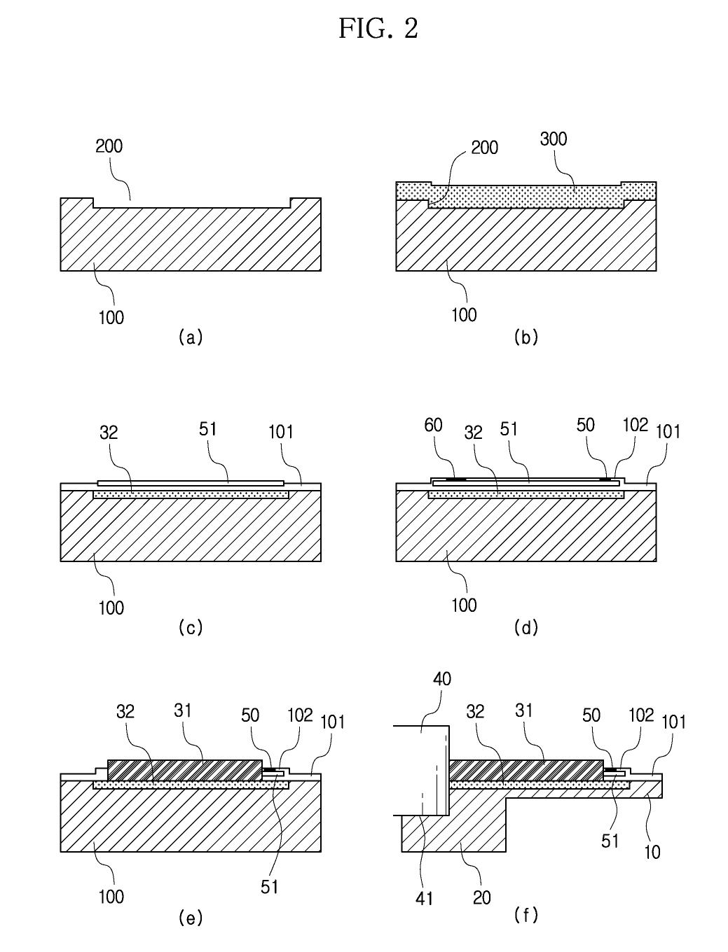

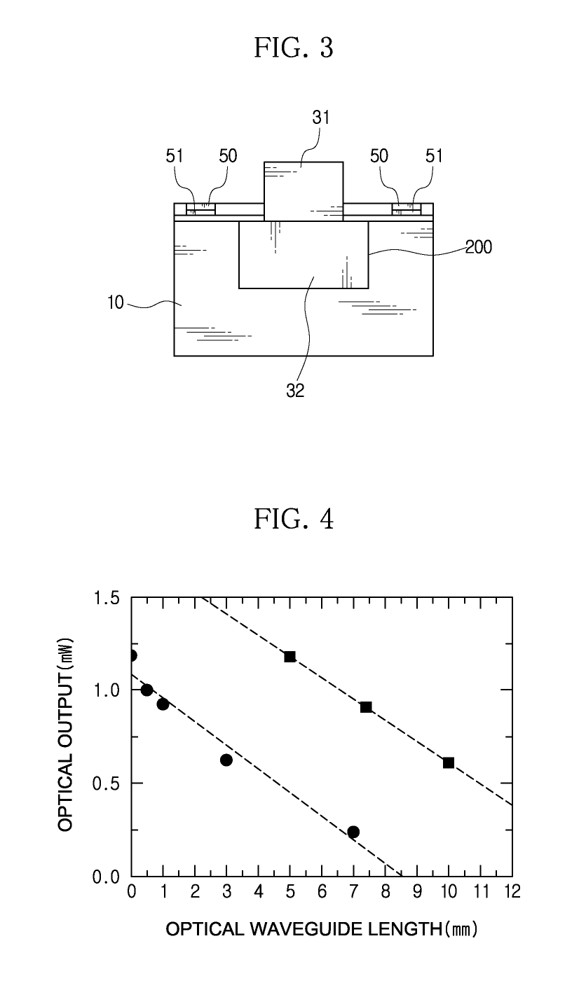

FIG. 1 is a perspective view illustrating a neural probe array 1 according to an exemplary embodiment, FIG. 2 is a diagram illustrating a process of manufacturing the neural probe array 1, and FIG. 3 is a cross-sectional view illustrating a stack structure of the neural probe array 1.

FIGS. 2 and 3 are cross-sectional views illustrating a manufacturing method and a structure of the neural probe array 1, and it should be understood that a specific part of FIG. 1 cut away is not drawn to scale.

As shown in FIGS. 1 through 3, the neural probe array 1 includes a probe body 10 that is implanted into a subject (not shown), a fixture body 20 to support a rear end of the probe body 10, a cladding 32 extending in a lengthwise direction of the probe body 10 in an upper part of the probe body 10, and an optical waveguide member 31 installed on the cladding 32 along the cladding 32.

An optical fiber 40 is seated in a groove 41 formed in the fixture body 20 at a rear part of the fixture body 20 and is fixed in the fixture body 20.

A rear end of the optical waveguide member 31 is formed in contact with a front end of the optical fiber 40. The rear end of the optical waveguide member 31 is aligned with the front end of the optical fiber 40 to allow light transmission.

A plurality of electrodes 50 is disposed on the probe body 10, and a plurality of pads 60 is disposed on the fixture body 20.

Each electrode 50 is electrically connected to each pad 60 via a signal line 51 installed through the probe body 10 and the fixture body 20. Each pad 60 is electrically connected to a printed circuit board (PCB) (not shown) to allow a neural response signal received from the electrode 50 to be transmitted to an external signal processing and analysis apparatus (not shown).

According to this construction, light for neural stimulation transmitted from an external light source (not shown) is transmitted to the optical waveguide member 31 through the optical fiber 40, and the transmitted light is guided through the optical waveguide member 31. In this instance, the cladding 32 disposed below the optical waveguide member 31 is made from a material having a different refractive index from the optical waveguide member 31. Accordingly, the light guided through the optical waveguide member 31 is totally reflected without leaking through a side of the optical waveguide member 31 by the cladding 32 or air surrounding the side of the optical waveguide member 31.

The light guided through the inside of the optical waveguide member 31 while undergoing total internal reflection is outputted through a front end of the optical waveguide member 31 to apply an optical stimulus to neurons.

A response signal of the optically stimulated neurons is received at the electrodes 50, and the signal processing and analysis apparatus may analyze the neural response signal received from the electrodes 50.

Hereinafter, with reference to FIG. 2, a method for manufacturing the neural probe array 1 according to this embodiment is first described, and then, a unique structure of the neural probe array 1 according to the manufacturing method is described.

As shown in FIG. 2(a), a Silicon On Insulator (SOI)-based first substrate 100 is formed, and a cavity 200 corresponding to a shape of a cladding 32 is formed in an upper part of the first substrate 100 through a deep reactive-ion etching (DRIE) process. According to this embodiment, a depth of the cavity 200 is 20 μm.

Subsequently, a flat plate-shaped second substrate 300 made from borosilicate glass having a thickness of 500 μm is formed. A softening point of the second glass substrate 300 is lower than that of the first SOI substrate 100.

Subsequently, in a vacuum condition, the second substrate 300 is put on the first substrate 100, and bonded to one another. According to this embodiment, the first substrate 100 and the second substrate 300 are strongly bonded to one another by anodic bonding that is a joining method by voltage.

As the first substrate 100 and the second substrate 300 are bonded, the cavity 200 formed in the upper part of the first substrate 100 is closed and sealed in a vacuum state by the second substrate 300.

Subsequently, the first substrate 100 and the second substrate 300 bonded to one another are put in a furnace (not shown) in a non-vacuum state, and are heated at temperature of 800° C. for 2 hours.

As the temperature of 800° C. above the softening temperature of the second glass substrate 300 is applied, the softened and melted second substrate 300 fills the inside of the cavity 200. In this instance, there is a predetermined pressure difference between the inside of the cavity 200 in a vacuum state and the outside of the cavity 200 in a non-vacuum state, and the second substrate 300 melted by the pressure difference between the inside and the outside of the cavity 200 is sucked into the cavity 200, thereby effectively filling the whole cavity 200 (FIG. 2(b)).

Subsequently, an unnecessary portion of the second substrate 300 except a portion filled in the cavity 200 is removed through a chemical mechanical planarization (CMP) process to form a cladding 32 embedded in the cavity 200 of the first substrate 100.

Subsequently, a SiO₂ insulation layer 101 having a thickness of 300 nm and a signal line 51 made of gold having a thickness of 300 nm are patterned on the first substrate 100 through a lift-off process around a region in which the cladding 32 is formed, an electrode 50 and an pad 60 made from iridium are attached onto the signal line 51, and a SiO₂ insulation layer 102 having a thickness of 400 nm is coated to protect the signal line 51 (FIG. 2(c) and FIG. 2(d)).

Subsequently, an optical waveguide member 31 made from SU-8 having a thickness of 15 μm is formed on the cladding 32 (FIG. 2(e)).

Finally, a groove 41 is formed at a rear end of the first substrate 100 through a DRIE process, and a front end of a lower part of the first substrate 100 is etched to form a shape of a fixture body 20 integrally formed with the probe body 10.

An optical fiber 40 is seated in the groove 41, and is aligned with the optical waveguide member 31 such that a front part of the optical fiber 40 comes in close contact with a rear part of the optical waveguide member 31, and then fixed to the optical waveguide member 31, as a consequence, the neural probe array 1 is completed.

As best shown in FIG. 3, the neural probe array 1 manufactured by the manufacturing method according to this embodiment is formed in such a structure that the cladding 32 allowing total internal reflection of light having passed through the optical waveguide member 31 is embedded in the recessed cavity 200 formed in the upper part of the probe body 10.

The cladding 32 according to this embodiment is formed to have the same height as the cavity 200, and thus, is completely embedded in the cavity 200 without protruding beyond a top of the probe body 10.

According to this construction, an overall height of the probe body 10 may be greatly reduced.

Also, because the height of the cladding 32 is limited only by the height of the probe body 10, a thickness of the cladding 32 may be increased by forming the cavity 200 at a maximum depth within a range allowed by the height of the probe body 10.

As described in the foregoing, as the cladding 32 increases in thickness, total internal reflection of light guided through the optical waveguide member 31 increases and a loss of light reduces, thereby increasing an output of light outputted from the optical waveguide member 31.

FIG. 4 is a graph illustrating a comparison of light transmission efficiency, i.e., an optical output value between the neural probe array described in the related art and the neural probe array 1 according to this embodiment. In the both neural probe arrays, the claddings have an equal thickness.

In FIG. 4, a line indicated by a square shows results of the neural probe array 1 according to this embodiment, and a line indicated by a circle shows results of the neural probe array according to the related art.

As shown in FIG. 4, in case the optical waveguide members have an equal length, it can be seen that the neural probe array 1 according to this embodiment has higher light transmission efficiency about four times than the related art.

Like this, using the structure of the neural probe array 1 with high light transmission efficiency, the neural probe array 1 capable of concurrent optical stimulation to multiple sites as well as one site may be formed.

Its description is provided with reference to FIG. 1 again.

As shown in FIG. 1, the neural probe array 1 according to this embodiment has four probe bodies 10 formed at the front end of one fixture body 20.

Through the process of FIG. 2(f) described in the foregoing, the four probe bodies 10 are integrally formed with the fixture body 20.

For each probe body 10, the plurality of electrodes 50 are formed, and each electrode 50 is electrically connected to the pads 60 of the fixture body 20 via the signal line 51.

As shown in FIG. 1, the optical waveguide member 31 extends as one strand from the front end of the optical fiber 40 and branches into two strands which in turn, branch into four strands, and each split strand extends to each probe body 10. A branching shape of the optical waveguide member 31 is not limited to the above shape, and may include any shape that extends as one strand from the front end of the optical fiber 40 and branches into a plurality of strands.

Although not shown minutely in FIG. 1, the cavity 200 is formed below the optical waveguide member 31 in the substantially same shape as the shape in which the optical waveguide member 31 extends. The cavity 200 is formed starting from the probe body 10 ending at the front end of the optical fiber 40 across an upper part of the fixture body 20. The cladding 32 is embedded in the cavity 200, and the optical waveguide member 31 is attached to the cladding 32.

That is, the optical waveguide member 31, the cladding 32, and the cavity 200 are formed in the substantially same shape in the probe body 10 and the fixture body 20, and a placement relation of the optical waveguide member 31, the cladding 32, and the cavity 200 is same as above.

Referring to FIG. 1, the signal line 51 extends across below the optical waveguide member 31 for a certain section to electrically connect to the pads 60 formed at both sides of the fixture body 20. Thus, for the certain section, the optical waveguide member 31 does not come in contact with the cladding 32 by the signal line 51, but because an area of a non-contact part is very small, a loss of light at the corresponding part is not too great.

As already known, while light propagates to the optical waveguide member 31 through the optical fiber 40, 80% or higher of light outputted from the optical fiber 40 is lost.

Accordingly, in the case of a neural probe array having a thin cladding like the related art, when a plurality of probe bodies are formed and an optical waveguide member branches into a plurality of strands, a loss of light further increases due to branching of light and optical stimulation of sufficient output is not achieved.

However, according to this embodiment, because light transmission efficiency is significantly improved as described in the foregoing, even in case the plurality of probe bodies 10 are formed and the optical waveguide member 31 branches into a plurality of strands corresponding thereto, a great loss of light does not occur, so the neural probe array 1 capable of stimulating multiple sites of neurons concurrently by the plurality of probe bodies 10 may be formed.

What is claimed is:

1. A neural probe array comprising:
a probe body that is implanted into a subject;
a fixture body to support a rear end of the probe body;
a cladding extending in a lengthwise direction of the probe body in an upper part of the probe body; and
an optical waveguide member installed on the cladding along the cladding,
wherein the cladding is embedded in a recessed cavity formed in the upper part of the probe body.

2. The neural probe array according to claim 1, further comprising:
an optical fiber fixed in the fixture body to transmit light to the optical waveguide member,
wherein the cavity is formed to extend to a front end of the optical fiber across an upper part of the fixture body so that the cladding and the optical waveguide member extend to the front end of the optical fiber, and
a rear end of the optical waveguide member is aligned with the front end of the optical fiber to allow light transmission with the optical fiber.

3. The neural probe array according to claim 2, wherein a plurality of probe bodies are formed in one fixture body, and
the optical waveguide member extends as one strand from the front end of the optical fiber and branches into a plurality of strands which extend to each of the plurality of probe bodies.

4. The neural probe array according to claim 1, wherein the cladding is completely embedded in the cavity without protruding beyond a top of the probe body.

5. The neural probe array according to claim 4, wherein the cladding is formed with a same height as the cavity.

6. A method for manufacturing the neural probe array defined in claim 1, the method comprising:
forming the cavity in an upper part of a first substrate;
embedding the cladding in the cavity;
forming the optical waveguide member on the cladding along the cladding; and
forming the probe body by cutting the first substrate off.

7. The method for manufacturing the neural probe array according to claim 6, wherein the embedding of the cladding in the cavity comprises:
forming a second substrate having a lower softening point than the first substrate;
bonding the second substrate onto the first substrate;
applying a higher temperature than the softening point of the second substrate to the first substrate and the second substrate to fill the cavity with the melted second substrate; and
removing the other portion than a portion of the second substrate filled in the cavity.

8. The method for manufacturing the neural probe array according to claim 7, wherein the bonding of the second substrate onto the first substrate is performed in a vacuum state so that the cavity is sealed in a vacuum state by the second substrate, and
the filling of the cavity with the second substrate is performed in a non-vacuum state, and the second substrate is sucked into the cavity by a pressure difference between an inside and an outside of the cavity.

9. The method for manufacturing the neural probe array according to claim 6, wherein the probe body and the fixture body are integrally formed by cutting the first substrate off,
the method for manufacturing the neural probe array further comprises:
forming, in the fixture body, a groove in which the optical fiber for transmitting light to the optical waveguide member is seated; and
attaching the optical fiber to the groove,
the cavity is formed to extend to a front end of the optical fiber across the upper part of the fixture body so that the cladding and the optical waveguide member extend to the front end of the optical fiber, and
a rear end of the optical waveguide member is aligned with the front end of the optical fiber to allow light transmission with the optical fiber

10. The method for manufacturing the neural probe array according to claim 6, wherein a plurality of probe bodies are formed for one fixture body, and
the optical waveguide member extends as one strand from the front end of the optical fiber and branches into a plurality of strands which extend to each of the plurality of probe bodies.
